(19) United States
(12) Patent Application Publication
(10) Pub. No.: US 2006/0100451 A1
(43) Pub. Date: May 11, 2006

(54) Process for manufacturing ethylene oxide

(76) Inventors: Christine Poulain, Carry le Rouet (FR); Mehdi Rghioui, Martigues (FR); Hassan Taheri, Naperville, IL (US)

Correspondence Address: NIXON & VANDERHYE, PC, 901 NORTH GLEBE ROAD, 11TH FLOOR, ARLINGTON, VA, 22203, US

(21) Appl. No.: 10/543,736

(22) PCT Filed: Jan. 21, 2004
(86) PCT No.: PCT/GB04/00188

§ 371 Date: Jul. 29, 2005

(51) Int. Cl. C07D 301/10

(52) U.S. Cl. 549/534

(57) ABSTRACT

The invention relates to a process for manufacturing ethylene oxide by the catalytic oxidation reaction of ethylene with molecular oxygen. The process comprises contacting a reactive gas mixture current comprising ethylene and molecular oxygen with a silver-based catalyst in the form of particles arranged as a fixed bed in reaction tubes combined as a bundle in a tube reactor. The process is characterized in that the reactive gas mixture current flowing through the reaction tubes is contacted with the catalyst particles diluted with particles of an inert solid in a proportion increasing in the flow direction of said current. Preferably, the dilution of the catalyst particles with those of the inert solid is performed over a portion of the catalyst particles arranged in a zone of the reaction tubes located towards the outlet of said tubes and more particularly extending up to the outlet of said tubes, in the flow direction of the reactive gas mixture current. The main objective of the invention is the improvement of the safety of the process as regards the explosion risks by deviating the reaction temperature in particular at the outlet of the reaction tubes from the flammability zone of the gas current.

The present invention relates to a process for manufacturing ethylene oxide by the catalytic oxidation reaction of ethylene.

The catalytic oxidation reaction of ethylene that leads to the formation of ethylene oxide is generally carried out by placing a reactive gas mixture current comprising ethylene and molecular oxygen in contact with a silver-based catalyst in the form of particles forming a fixed bed in reaction tubes combined as a bundle in a-tube reactor. The reaction is known to be strongly exothermic.

Several problems arise simultaneously in the manufacture of ethylene oxide. The most serious problems are linked to the strongly exothermic character of the reaction and to the control of the temperature of the reaction, in particular the whole length of the reaction tubes, from the entry of the reactive gas mixture into the tube reactor up to the exit of the current resulting from the reaction. One of the major risks of the process is the formation of hot spots leading to reaction runaways, known generally under the term “post-combustion”. In parallel with the formation of ethylene oxide, secondary reactions can develop, in particular reactions involving complete oxidation of the ethylene or of the ethylene oxide into carbon dioxide and water, a reaction involving partial oxidation of the ethylene into formaldehyde, and a reaction involving isomerisation of the ethylene oxide into acetaldehyde, the majority of said secondary reactions being promoted by an increase in the temperature. A reaction temperature profile which is irregular, uncontrolled and in particular increasing the whole length of the reaction tubes can lead not only to hot spots, but also an excessive final temperature. The hot spots and an excessive final temperature affect the selectivity of the reaction to ethylene oxide and the formation of secondary products that are then difficult to separate from the ethylene oxide. In addition, locally elevated temperatures and an excessive final temperature can rapidly attain values which would lead the process into the flammability zone of the reactive gas mixture current and thus cause an explosion.

Solutions have been proposed for partially resolving some of said problems through methods of various degrees of complexity. In Australian patent. AU 211 242, a process is proposed for manufacturing ethylene oxide by passing a reactive gas mixture current comprising ethylene and oxygen into reaction tubes containing particles of various silver-based catalysts deposited on an alumina support. The latter are arranged along the reaction tubes with an increasing concentration of silver in the flow direction of said current. The various catalysts are thus arranged so that the catalytic activity is increasing in the flow direction of the reactive gas mixture current. In comparative tests, all of the particles of the various catalysts were diluted in a constant proportion along the reaction tubes with particles of an inert solid, in particular with the alumina support that had served for the preparation of said catalysts. As a result of said mixture, the quantity of ethylene oxide produced greatly decreased, despite the use of various catalysts with an increasing concentration of silver.

In American patent U.S. Pat. No. 3,147,084, a tube reactor intended for catalytic chemical reactions such as catalytic olefin oxidations is proposed. The reactor comprises a reaction zone followed by a cooling zone, the two zones being passed through by a bundle of reaction tubes filled with a solid catalyst. The catalytic reaction develops by the passage of a reactive current circulating in the reaction tubes and flowing successively through the reaction zone and then into the cooling zone. The portion of the reaction tubes corresponding to the cooling zone may be empty, or may contain the catalyst, or else may be filled with solid materials such as metallic particles capable of conducting heat and of creating a multitude of passages through which the reactive gas mixture current flows.

In American patent U.S. Pat. No. 4,061,659, there is proposed, with the aim of reducing the reaction involving the isomerisation of ethylene oxide into acetaldehyde, a process for manufacturing ethylene oxide by placing a reactive gas mixture current comprising ethylene and oxygen in contact with a silver-based catalyst arranged in reaction tubes. Each of said tubes comprises first of all a reaction zone containing the catalyst in the form of particles, and then a cooling zone containing particles of an inert solid such as an alumina and having a small specific surface area, in particular of less than 0.1 m²/g. It is also proposed, in particular in order to avoid the formation of hot spots, that the catalyst be used, in the reaction zone of the reaction tubes, in the form of particles mixed with particles of an inert solid diluent (such as the inert solid mentioned above), so that the concentration of catalyst increases in the flow direction of the reactive gas mixture current in the reaction tubes.

In American patent U.S. Pat. No. 4,921,681, a process for manufacturing ethylene oxide substantially similar to that described above is proposed, except that it does not suggest mixing the catalyst particles with particles of an inert solid diluent, but only using a zone for cooling of the reaction tubes which is filled with particles of an inert solid and in certain cases terminates in a completely empty section devoid of any particles.

None of the above proposals is perfectly satisfactory, in particular for the manufacture of ethylene oxide on an industrial scale, and it has even been found that some of said proposals conversely aggravate the technical problems mentioned above, in particular the problems of reaction runaways, hot spots and explosion risks.

The process of the present invention is intended to resolve the technical problems described above. It is intended in particular to improve the safety of the process more particularly as regards the risks of reaction runaways, hot spots and explosion, by controlling in particular the reaction temperature profile, in particular from the inlet up to the outlet of the reaction tubes, and preferably in the zone extending towards the outlet of the reaction tubes.

The present invention relates to a process for manufacturing ethylene oxide by catalytic oxidation reaction of ethylene with molecular oxygen, said process comprising contacting a reactive gas mixture current comprising ethylene and molecular oxygen with a silver-based catalyst in the form of particles arranged in a fixed bed in reaction tubes combined as a bundle in a tube reactor, and being characterised in that the reactive gas mixture current flowing through the reaction tubes is contacted with the catalyst particles diluted with particles of an inert solid in a proportion increasing in the flow direction of said current.

By “catalyst particles diluted with particles of an inert solid” is meant in general catalyst particles mixed with particles of an inert solid diluent. The mixture resulting from the dilution of the catalyst with the inert solid occurs in particular in the form of a mixture of particles of the two solids used (the catalyst and the inert solid). By “dilution of the catalyst with the inert solid” is meant in general a dilution (or a mixture) of the catalyst particles with the particles of the,inert solid. Finally, there is meant in general by “inert solid” a solid compound that is substantially inert with respect to the products involved and formed in the manufacture of the ethylene oxide.

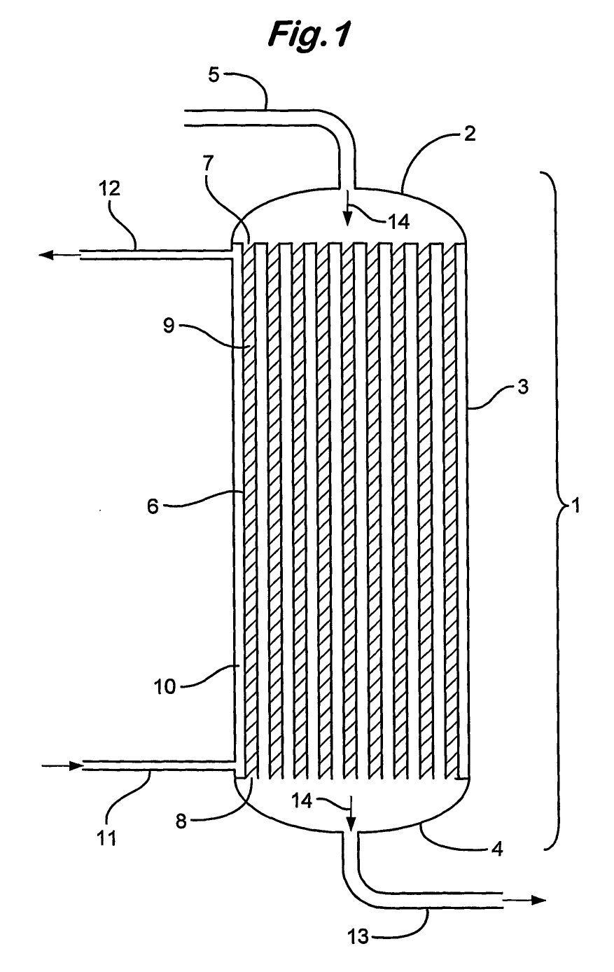

FIG. 1 represents diagrammatically a tube reactor comprising reaction tubes combined as a bundle and filled with a silver-based catalyst for the manufacture of ethylene oxide.

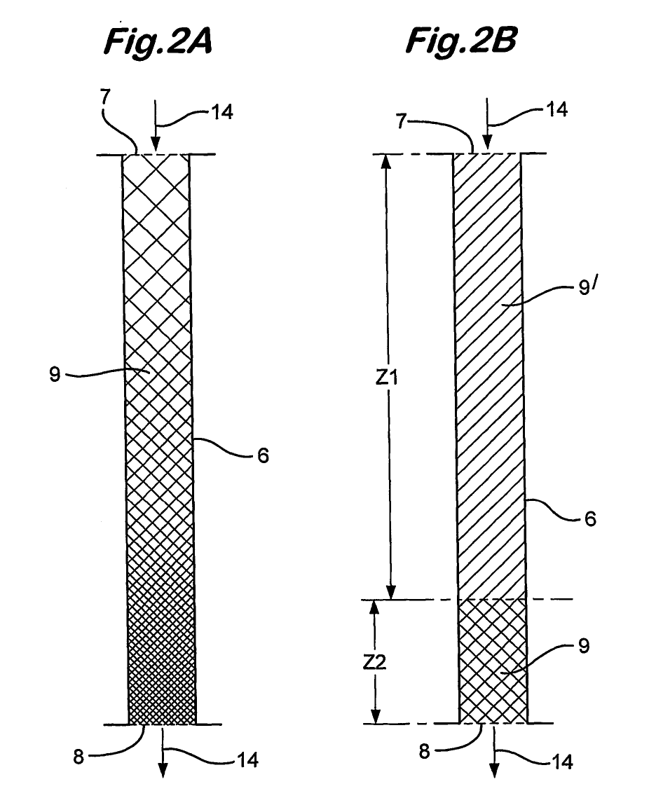

FIGS. 2_{A} and 2_{B} represent diagrammatically reaction tubes filled with a silver-based catalyst in the form of particles diluted respectively completely or partly with particles of an inert solid, according to the process of the invention.

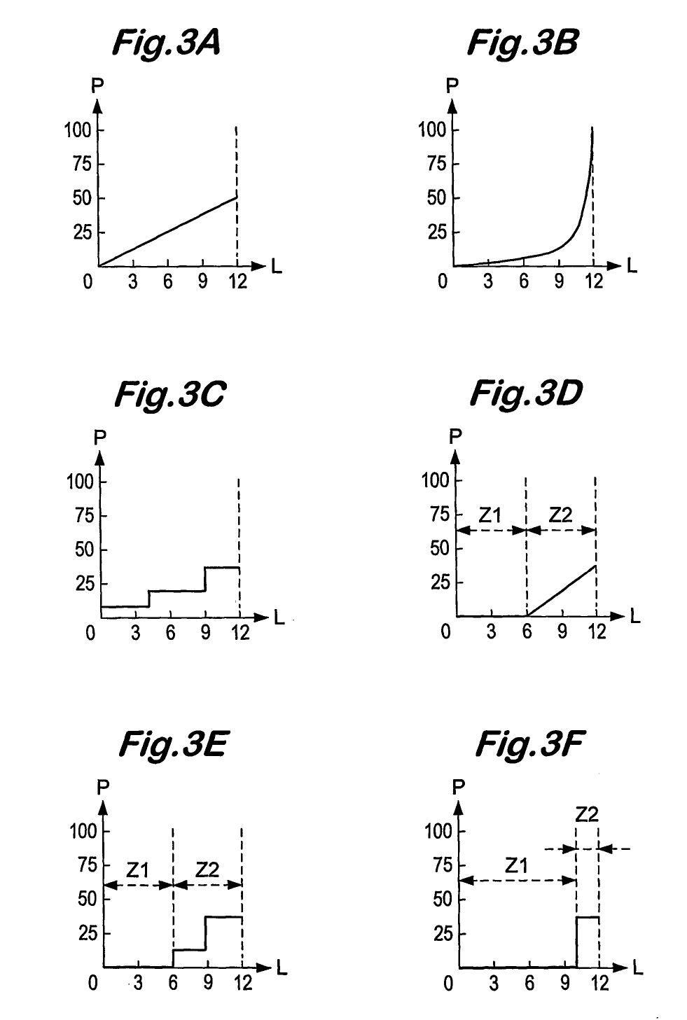

FIGS. 3_{A} to 3_{F} represent graphs linking, on the ordinate, the proportion (P) of particles of an inert solid in the mixture resulting from the dilution of the particles of the catalyst with those of said solid with, on the abscissa; the length L of the reaction tube measured from the inlet of the reaction tube, in the flow direction of the reactive gas mixture current.

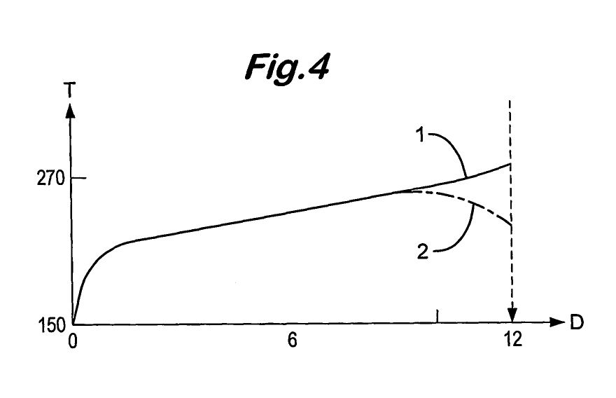

FIG. 4 represents a graph linking, on the ordinate, the temperature T of the reactive gas mixture current measured along the reaction tubes with, on the abscissa, the distance D separating the point of the measurement of the temperature T from the inlet of the reaction tubes.

Owing to the invention, it was found that it is possible to obtain a reaction temperature profile which is relatively stable over the major part of the length of the reaction tubes and in particular which is substantially decreasing towards the outlet of the reaction tubes, with the aim in particular of avoiding hot spots and reaction runaways, reducing significantly the final temperature of the reaction and finally improving substantially the safety of the process. Such results are obtained more particularly because of the fact that the particles of the catalyst are diluted with particles of an inert solid in an increasing proportion in the flow direction of the reactive gas mixture current.

The proportion of particles of the inert solid in the mixture resulting from the dilution of the particles of the catalyst with those of said solid can increase continuously, for example according to a linear or exponential mode, or preferably discontinuously, in particular by one or more successive stages, in the flow direction of the reactive gas mixture current in the reaction tubes. The dilution of the particles of the catalyst with those of the inert solid can be carried out over all of the particles of the catalyst that are contained in the reaction tubes or, preferably, over a portion of the particles of the catalyst, said portion being arranged in a zone situated towards the outlet of the reaction tubes and more particularly in a final zone extending up to the outlet of the reaction tubes (in the flow direction of the reactive gas mixture current). Thus, it is preferred that the first zone of the reaction tubes, which is situated towards the inlet of the reaction tubes, contains the catalyst in the form of non-diluted particles, while the second zone, which immediately follows the first zone in the flow direction of the reactive gas mixture current, contains the catalyst in the form of particles diluted with the particles of the inert solid. By “catalyst in the form of non-diluted particles” is meant generally a silver-based catalyst in the form of particles not diluted with any particles of an inert solid and in particular not diluted with the particles of the inert solid. The silver-based catalyst in particular is involved, in particular as it is prepared and as it is used in the form of particles not mixed with particles of any inert solid diluent. In every case, it is particularly advantageous that the catalyst used in the form of particles thus diluted with the particles of the inert solid occupies at least the second zone situated towards the outlet of the reaction tubes (in the flow direction of the reactive gas mixture current) and in particular the final zone of the reaction tubes extending up to the outlet of the reaction tubes.

According to a preferred variant of the process according to the invention, the reactive gas mixture current flowing through the reaction tubes is placed in contact first of all with the catalyst in the form of non-diluted particles (more particularly not diluted with the particles of the inert solid) and arranged in a first zone Z1 of the reaction tubes which is situated towards the inlet of the reaction tubes, then with the catalyst in the form of particles diluted with the particles of the inert solid in the flow direction of said current, the particles thus diluted being arranged in a second tone Z2 of the reaction tubes, adjacent to the first zone Z1 and situated towards the outlet of the reaction tubes, preferably extending up to the outlet of the reaction tubes. The proportion of particles of the inert solid in the mixture resulting from the dilution of the particles of the catalyst with those of said solid can be with advantage constant over the whole of zone Z2 of the reaction tubes extending towards the outlet or, preferably, up to the outlet of the reaction tubes. According to another variant, the proportion of particles of the inert solid can also increase continuously or, preferably, discontinuously, in particular by one or more stages, in the flow direction of the reactive gas mixture current, over the whole of zone Z2 of the reaction tubes, extending more particularly towards the outlet or, preferably, up to the outlet of the reaction tubes.

The effects sought by the present invention are particularly remarkable in certain conditions and in particular in the following conditions. The zone Z2 of the reaction tubes in which the particles of the catalyst are more particularly diluted with the particles of the inert solid in constant or increasing proportion can commence in the last half of the length of the reaction tubes which is situated towards the outlet of the reaction tubes (in the flow direction of the reactive gas mixture current), preferably in the last third or the last quarter or else the last fifth of the length of the reaction tubes, situated towards the outlet of the reaction tubes, and in all cases at the latest before; the last thirtieth or preferably the last twenty-fifth of the length of the reaction tubes, situated towards the outlet of the reaction tubes. The zone Z2 of the reaction tubes can, preferably, extend into the final zone of the reaction tubes extending up to the outlet of the reaction tubes, so that the catalyst particles diluted with the particles of the inert solid occupy the whole of the final zone of the reaction tubes.

The proportion of the particles of the inert solid in the mixture resulting from the dilution of the particles of the catalyst with those of said solid can be in particular a proportion (P) expressed in volume of the particles of the inert solid in said mixture (volume measured as a bulk or apparent volume in standard temperature and pressure conditions). The proportion (P) can be more particularly such that the number of parts by volume of the particles of the inert solid is chosen in a range extending from 1 to 99 parts, preferably from 1 to 75 parts, particularly from 2 to 50 parts, more particularly from 2 to 40 parts or else from 5 to 35 parts per 100 parts by volume of said mixture. The proportion (P) can be more particularly chosen in a range extending from 1 to 99 parts, more particularly from 1 to 75 parts by volume of the particles of the inert solid per 100 parts by volume of the mixture resulting from the dilution of the particles of the catalyst with those of said solid, in particular when the dilution of the catalyst is carried out over the whole of the particles of the catalyst that are contained in the reaction tubes, or else when a portion of the particles of the catalyst is diluted with the particles of the inert solid in the zone Z2 of the reaction tubes which commences in the second half or the last third of the length of the reaction tubes situated towards the outlet of the reaction tubes. The proportion (P) can be, preferably, chosen in a range extending from 2 to 50 parts, more particularly from 2 to 40 parts or else from 5 to 35 parts by volume of the particles of the inert solid per 100 parts by volume of the mixture resulting from the dilution of the particles of the catalyst with those of said solid, in particular when a portion of the particles of the catalyst is diluted with the particles of the inert solid in the zone Z2 of the reaction tubes which commences in the last quarter or the last fifth of the length of the reaction tubes, situated towards the outlet of the reaction tubes.

The tube reactor is generally of the vertical shell-and-tube exchanger type, that is to say comprising a vertical bundle of reaction tubes. By “bundle of reaction tubes” is generally meant an assemblage of reaction tubes identical and parallel with one another. The tube reactor can generally comprise three successive and adjacent chambers through which flows the reactive gas mixture current:
an inlet chamber of the reactive gas mixture current,
then a central chamber comprising the bundle of reaction tubes and in which there forms a gas mixture current containing the ethylene oxide resulting from the catalytic oxidation reaction of the ethylene with the molecular oxygen, and
an outlet chamber of the gas mixture current containing the ethylene oxide.

The central chamber comprises generally a bundle of reaction tubes immersed in a heat exchange fluid and filled with the silver-based catalyst in the form of particles at least partly diluted according to the invention with the particles of the inert solid. The reactive gas mixture current passes to the interior of the reaction tubes and forms the ethylene oxide by contact with the catalyst. Each reactor tube of the bundle generally comprises an inlet issuing into the inlet chamber and an outlet issuing into the outlet chamber of the tube reactor. The reaction tubes have generally a cylindrical form and can have a length (L) of from 6 to 20 m, preferably of from 8 to 15 m, and an internal diameter (Di) which can be chosen in a range of from 12 to 100 mm, preferably from 20 to 80 mm.

The silver-based catalyst can be chosen from among the silver-based catalysts capable of catalysing the oxidation reaction of ethylene to ethylene oxide with the aid of molecular oxygen. The catalyst is preferably a silver-based supported catalyst, in particular comprising metallic silver deposited on a solid support, preferably on a refractory and more particularly porous solid support. The support can be chosen from among refractory products of natural, artificial or synthetic origin, preferably from among those having a macro-porous structure, more particularly having a specific surface area (B.E.T) of less than 20 m²/g, in particular of from 0.01 to 10²m/g, and an apparent porosity of more than 20% by volume, more particularly of from 30 to 70% by volume. The most appropriate supports can be those that comprise siliceous and/or aluminous products (based on silica and/or alumina respectively). For example, the supports can be chosen from among the oxides of aluminium (more particularly those known under the trade reference “Alundum”®, charcoal, pumice stone, magnesia, zirconia, kieselguhr, fuller's earth, silicon carbide, porous agglomerates containing silicon and/or silicon carbide, clays, natural, artificial or synthetic zeolites, metal oxide gel-based materials containing oxides of heavy metals such as molybdenum or tungsten, and ceramic products. Aluminous products are preferred, in particular those containing alpha type aluminium, having in particular a specific surface area (B.E.T.) of from 0.15 to 0.6 m²/g and an apparent porosity of from 46 to 52 % by volume. The B.E.T. method used to determine the specific surface area is described in J. Am. Chem. Soc., 60, 309-16 (1938).

The catalyst can contain from 2 to 25%, preferably from 5 to 20% by weight of silver. It can in addition contain at least one metallic promoter agent, in particular chosen from among the alkaline metals, alkaline-earth metals such as calcium or barium, and other metals such as thallium, antimony, tin or rhenium. The catalyst can be in the form of particles having in particular a mean size at least equal to 1 or 2 mm and at most equal to half the internal diameter of the reaction tubes employed, in particular a mean size chosen from a range of from 1 to 20 mm, preferably from 3 to 12 mm, for example in the form of spherical, hemispherical, spheroidal, cylindrical particles, rings, pellets or granules. The catalyst can be prepared according to various processes such as those described in the American patents U.S. Pat. No. 3,043,854, U.S. Pat. No. 3,207,700, U.S. Pat. No. 3,575,888, U.S. Pat. No. 3,702,259 and U.S. Pat. No. 3,725,307, and in the European patent EP 0 266 015. The catalyst used in the process of the present invention can have with advantage a constant and uniform content of silver, whatever the reaction tubes of the reactor may be, and/or whatever the form of the catalyst may be, namely in the form of particles diluted or not diluted with the particles of the inert solid.

One of the advantages of the present invention is being able to use a fixed bed containing the catalyst over the whole (or almost the whole) of the length of the reaction tubes, from the inlet up to the outlet of the reaction tubes, and more particularly in the zone situated towards the outlet. The outlet of the reaction tubes can generally comprise a device for supporting the fixed bed. In this case, it is possible for the support device to be filled at least in part with the catalyst in the form of particles diluted with the particles of the inert solid. Thus, owing to the dilution of the particles of the catalyst with the particles of the inert solid, a maximum charge of the fixed bed containing the catalyst can be applied per internal tube volume available in the reactor, said charge being more particularly capable of being active in the production of ethylene oxide. These advantageous results can be simultaneously obtained with a reaction temperature profile relatively stable over the major part of the length of the reaction tubes and comprising in particular a substantially decreasing profile towards the outlet of the reaction tubes.

The inert solid can be chosen from among solid compounds inert or substantially inert with respect to the products involved and formed in the manufacture of the ethylene oxide. The inert solid can be in the form of particles, more particularly of spherical, hemispherical, spheroidal, cylindrical particles, rings, pellets or granules, in particular in the form of particles such that a fixed bed formed with said particles exhibits a low pressure drop, more particularly a pressure drop identical to or preferably less than that of an identical fixed bed but one formed with the particles of the catalyst. The mean size of the particles of the inert solid can be chosen in a range of from 1 to 20 mm, preferably from 3 to 12 mm, and more particularly can be identical to that of the particles of the catalyst. The inert solid can be chosen from among metals, metal alloys and refractory products, more particularly products used as inert filling solids. Preferably, it can be chosen from among refractory products of a nature different or preferably identical to that of the solid support used in the preparation of the catalyst. The inert solid can be chosen in particular from among the catalyst supports, more particularly those mentioned above. It can more particularly be chosen from among refractory products, preferably from among refractory oxides, refractory clays, ceramic products and glass type materials, more particularly those based on sodium polysilicates containing, for example, a stoichiometric excess of silica. The inert solid can with advantage be in the form of particles, in particular particles of a refractory product having a small B.E.T. specific surface area, preferably of less than 0.1, more particularly less than 0.05, in particular less than 0.01 m²/g. The inert solid can be, for example, chosen from among silica, alumina, alumino-silicates, silico-aluminates, clays, magnesite, dolomite, magnesia, zirconia, calcium oxide, silicon carbide, mixtures of alumina and silica optionally modified by alkaline or alkaline-earth metals. More particularly, the inert solid can be in the form of particles having a nature, a shape and a mean size similar to or preferably identical to those of the catalyst support.

The process for manufacturing ethylene oxide employs molecular oxygen, which may be used in the form of pure molecular oxygen, for example with an oxygen purity equal to or more than 95% by volume, or in the form of air. The reactive gas mixture current which flows through the reaction tubes of the reactor may consist of a gaseous mixture of ethylene, molecular oxygen and optionally one or more other gases chosen from among carbon dioxide, nitrogen, argon, methane, ethane and at least one reaction inhibitor (or moderator) chosen in particular from among halogenated hydrocarbons such as ethyl chloride, vinyl chloride or 1,2-dichloroethane. In the reactive gas mixture current, the concentration of ethylene is generally as high as possible, more particularly equal to or less than 40% by volume, and it is in particular chosen from a range of from 15 to 35% by volume. The concentration of molecular oxygen in the reactive gas mixture current can be chosen from a range of from 3 to 12%, preferably from 4 to 10% by volume. The concentration of carbon dioxide in the reactive gas mixture current is generally less than or equal to 10% by volume, and can be chosen from a range of from 4 to 8% by volume. Methane and/or nitrogen can be used as diluents in the reactive gas mixture current in order more particularly to reduce the flammability zone of the gas current and to move it towards a more distant, non-used zone. Thus methane and/or nitrogen can be present in the reactive gas mixture current in a concentration as high as possible. For example, the reactive gas mixture current can contain by volume from 15 to 40% of ethylene, from 3 to 12% of molecular oxygen, from 0 to 10% of carbon dioxide, from 0 to 3% of ethane, from 0.3 to 50 parts by volume per million (vpm) of a reaction inhibitor (or moderator) of the halogenated hydrocarbon type, the remainder being argon and/or nitrogen and/or methane. The absolute pressure of the reactive gas mixture current in the reaction tubes can be chosen in a range of from 0.1 to 4 MPa, preferably from 1 to 3 MPa. The volume space hour velocity (VSHV) of the reactive gas mixture current in the reaction tubes can be chosen in a range of from 1000 to 10 000 h⁻¹ (m³/.h of gas per m³ catalyst), preferably from 2000 to 8000 h⁻¹, measured in standard temperature and pressure conditions.

The reactive gas mixture current prior to flowing in the reaction tubes can be advantageously pre-heated to a temperature of from 100 to 200° C., preferably from 140 to 190° C. The temperature of the reactive gas mixture current in the reaction tubes can be chosen in a range of from 140 to 350° C., preferably from 180 to 300° C., more particularly from 190 to 280° C. The temperature of the reactive gas mixture current at the inlet of the reaction tubes can rise very rapidly up to a temperature equal to or more than 210° C. It can then, owing to the process of the invention, continue to increase, but far more moderately, and attain a maximum temperature at most equal to 270° C., preferably at most equal to 265° C., more particularly at most equal to 260° C. or even to 255° C., in particular over a portion of the length of the reaction tubes lying between the third and fifth sixths, preferably between the fourth and fifth sixths of the length of the reaction tubes, counting from the inlet of the reaction tubes (in the flow direction of the reactive gas mixture current). At the outlet of the reaction tubes, the temperature of the gas current resulting from the reaction can remain at said maximum temperature or, preferably, can decrease substantially to a temperature equal to or less than 250° C., preferably equal to or less than 240° C., in particular equal to or less than 230° C., and more particularly equal to or less than 220° C., for example in a range extending from 180′ to 250° C., preferably from 190 to 240° C., in particular from 200 to 230° C., and more particularly from 200 to 220° C.

It is particularly advantageous to note that, owing to the process of the invention, the exchange of heat along the reaction tubes makes it possible to combine, on the one hand, a reaction temperature profile which is relatively stable over the majority of the length of the reaction tubes, and which terminates in a substantial reduction in the temperature towards the outlet of the reaction tubes, with, on the other hand, a maximum charge of the fixed bed containing the catalyst, said charge being used in conditions of optimum activity per unit of internal tube volume available in the reactor. This makes it possible to prevent a not inconsiderable portion of the reaction tubes being sacrificed to an objective other than the production of ethylene oxide. One of the other major advantages of the process of the invention comes from the fact that the temperature of the reactive gas mixture current at the outlet of the reaction tubes is reached after a substantially decreasing profile and that it can be significantly reduced, for example by at least 3° C. or even 5° C., compared with that of the conventional processes. The result of this is that, all other conditions being equal, in particular an identical concentration of molecular oxygen in the gas current, the distance of the reaction temperature from the flammability zone of said current may be substantially increased and thus make it possible to provide a far safer process without losing an excessive part of the production of ethylene oxide.

The bundle of reaction tubes may be immersed in a heat exchange fluid chosen in particular from among organic heat carrying fluids and water at saturation temperature under pressure. The organic heat carrying fluids may be mixtures of oils or hydrocarbons such as linear or branched alkanes having in particular a boiling point higher than the maximum reaction temperature. It is possible to use the organic heat carrying fluids at a relative pressure of from 100 to 1500 kPa, preferably from 200 to 800 kPa, more particularly from 200 to 600 kPa. The organic heat carrying fluids may be chosen in particular from “isopar”® of Exxon, “Therminol”® of Monsanto and “Dowtherm”® of Dow Chemicals. They may be used according to a process and a heat exchange apparatus such as those described in European patent application EP 0 821 678, in particular in FIG. 1 or 2, or in American patent U.S. Pat. No. 4,759,313. The heat exchange fluid may also be water at saturation temperature under pressure, in particular at a relative pressure of from 1500 to 8000 kPa. In this case, the water at saturation temperature under pressure may be used according to a process and a heat exchange apparatus such as those described in American patent U.S. Pat. No. 5,292,904. The temperature of the heat exchange fluid at the outlet of the tube reactor generally lies between 210 and 300° C., preferably between 220 and 280° C., more particularly between 210 and 280° C. The temperature of the beat exchange fluid at the inlet of the tube reactor generally lies between 120 and 250° C., preferably between 130 and 240° C., more particularly between 130 and 230° C.

The process of the invention may with advantage be carried out continuously, more particularly by utilising continuously the reactive gas mixture current that flows through the reaction tubes and by recovering continuously at the outlet of the reactor the gas current resulting from the reaction and containing the ethylene oxide.

FIG. 1 is a diagrammatic representation of a tube reactor (1) capable of being used in the process for manufacturing ethylene oxide according to the invention. The tube reactor (1) is of the vertical shell-and-tube exchanger type. It comprises three successive and adjacent chambers: an inlet chamber (2), then a central chamber (3) and an outlet chamber (4). There issues into the inlet chamber (2) a pipe (5) for the feeding of a reactive gas mixture current containing ethylene and molecular oxygen. The central chamber (3) comprises a bundle of reaction tubes (6) parallel and identical to one another, and preferably cylindrical, each reaction tube (6) containing in inlet (7) issuing into the inlet chamber (2) and an outlet (8) issuing into the outlet chamber (4). The reaction tubes (6) are filled with a silver-based catalyst (9) in the form of particles partly diluted according to the invention with particles of an inert solid. The reaction tubes (6) are immersed in a beat exchange fluid (10) which is introduced into the central chamber (3) through a feed pipe (11) and which is withdrawn from the central chamber (3) through an extraction pipe (12). The outlet chamber (4) is provided with a pipe (13) for extraction of the gas current containing the ethylene oxide resulting from the reaction.

FIGS. 2_{A} and ²_{B} are diagrammatic representations of a reaction tube (6) used in the tube reactor (1) as shown in FIG. 1 and enabling the process of the invention to be carried out. The elements of FIGS. 2_{A} and ²_{B} identical to those shown in FIG. 1 are marked with the same numerical references. FIGS. 2_{A} and 2_{B} represent diagrammatically a reaction tube (6) which is provided with an inlet (7) and an outlet (8). In FIG. 2_{A}, the reaction tube (6) is filled in its entirety, that is to say from the inlet (7) up to the outlet (8) of the reaction tube, with a silver-based catalyst (9) in the form of particles diluted with particles of an inert solid in increasing proportions in the flow direction of the reactive gas mixture current (14).

In FIG. 2_{B}, the tube (6) is filled first of all in a first zone Z1 situated towards the inlet (7) of the reaction tube with a silver-based catalyst (9′) in the form of non-diluted particles (more particularly not diluted with particles of an inert solid), then in a second zone Z2 (adjacent to the first zone Z1 and extending up to the outlet (8) of the reaction tube) with a silver-based catalyst (9) in the form of particles diluted with particles of an inert solid in a proportion which is constant or increasing in the flow direction (14) of the reactive gas mixture current.

FIGS. 3_{A} to 3_{F} represent graphs linking, on the ordinate, the proportion (P) by bulk volume of particles of the inert solid in the mixture resulting from the dilution of the particles of the catalyst with those of said solid with, on the abscissa, the length (L) of the reaction tube (measured in metres from the inlet of the reaction tube, in the flow direction of the reactive gas mixture current), the total length of the reaction tube between the inlet and the outlet being equal to 12 m.

In FIG. 3_{A}, all of the particles of the catalyst are diluted with the particles of the inert solid in an increasing proportion (P), the increase in ()) being continuous and linear from the inlet (L=0 m) up to the outlet (L=12 m) of the reaction tube, in the flow direction of the reactive gas mixture current.

In FIG. 3_{B}, all of the particles of the catalyst are diluted with the particles of the inert solid as in FIG. 3_{A}, except that the proportion (P) of particles of the inert solid increases continuously and exponentially between the inlet (L=0 m) and the outlet (L=12 m) of the reaction tube.

In FIG. 3_{C}, all of the particles of the catalyst are diluted with the particles of the inert solid as in FIG. 3_{A}, except that the proportion (P) of particles of the inert solid increases discontinuously, more particularly in two successive stages in the flow direction of the reactive gas mixture current.

In FIG. 3_{D}, the particles of the catalyst are diluted with the particles of the inert solid in an increasing proportion (P), the increase in (P) being discontinuous between the inlet (L=0 m) and the outlet (L=12 in) of the reaction tube, in the-flow direction of the reactive gas mixture current. In a first zone Z1 extending from the inlet (L=0 in) of the reaction tube to L=6 in, the particles of the catalyst are not diluted (more particularly with those of the inert solid), so that the proportion (P) of particles of the inert solid is equal to 0 in said zone. In a second zone Z2 extending from L=6 in to the outlet (L=12 in) of the reaction tube, the particles of the catalyst are diluted with the particles of the inert solid in an increasing proportion (P), the increase in (P) being continuous and linear in said zone.

In FIG. 3_{E}, the particles of the catalyst are diluted with the particles of the inert solid in an increasing proportion (P), the increase in (P) being discontinuous as in FIG. 3_{D}, except that in the zone Z2 the particles of the catalyst are diluted with the particles of the inert solid in an increasing proportion (P), the increase in (P) being discontinuous, more particularly by a stage, in said zone.

In FIG. 3_{F}, the particles of the catalyst are diluted with the particles of the inert solid in an increasing proportion (P), the increase in (P) being discontinuous as in FIG. 3_{E}, except that the zone Z1 extends from the inlet (L=0 m) of the reaction tube to L=10 m, that the zone Z2 extends from L=10 m to the outlet (L=12 m) of the reaction tube, and that in the zone Z2 the particles of the catalyst are diluted with the particles of the inert solid in a constant proportion (P).

FIG. 4 represents a graph linking, on the ordinate, the temperature T (in degrees Celsius) of the reactive gas mixture current measured along the reaction tubes with, on the abscissa, the distance D (measured in metres) separating the point of the measurement of the temperature T from the inlet of the reaction tubes, the total length of the reaction tube between the inlet and the outlet being equal to 12 m. The curve (1) is that generally obtained (as a comparative example) when the catalyst is used as such, in the form of non-diluted particles (more particularly not diluted with particles of an inert solid), over the whole length of the reaction tubes, while the curve (2) is that obtained according to the invention, when the catalyst is used in the form of particles diluted with particles of an inert solid, more particularly in the second zone Z2 of the reaction tubes situated towards the outlet and preferably extending up to the outlet of the reaction tubes.

The process of the invention offers various advantages and more particularly the following advantages. The first and the most important of the advantages is that linked to the considerable improvement in the safety of the process, more particularly as regards the explosion risks. Owing to the process of the invention, in fact, the temperature of the reactive gas mixture current generally exhibits a substantially decreasing profile in the zone situated towards the outlet of the reaction tubes and more especially in the final zone extending up to the outlet of the reaction tubes. Because of this, the temperature of the reactive gas mixture current at the outlet of the reaction tubes deviates very substantially from the flammability zone of the gas current. As a result, the risk of the formation of hot spots and reaction runaways diminishes very notably, and the safety of the process in terms of the explosion risks is improved considerably. Another not inconsiderable advantage is linked to the fact that owing to the substantially decreasing temperature profile generally observed in the zone situated towards the outlet of the tubes, a significantly extended margin in the handling of the reaction temperature in terms of the flammability zone is now obtained. Said margin is sufficient in most cases for the operating conditions of the process to be able to be modified, for example for the concentration of molecular oxygen in the reactive gas mixture current to be increased in order to compensate at least in part for the slight drop in production of ethylene oxide generally observed when the catalyst particles are diluted with those of an inert solid. Said compensation may thus take place with advantage without jeopardising the substantial increase in safety attained more particularly in terms of the explosion risks. This is possible in particular by virtue of a substantially decreasing reaction temperature profile that is generally observed in the zone situated towards the outlet of the reaction tubes. Another advantage comes from the fact that the selectivity of the reaction to ethylene oxide is also improved. Finally, another advantage can also arise given that, owing to the dilution of the particles of the catalyst with those of the inert solid, it is possible to fill the reaction tubes over the whole (or almost the whole) of their length with the catalyst and/or with the mixture of the particles of the catalyst and the inert solid. This can be achieved without leaving, in particular at the start and/or at the end of the reaction tubes, empty zones or zones occupied with particles of an inert solid completely devoid of catalyst, with the sole aim of improving the heat exchange. Because of this, it is possible to achieve a maximum charge of fixed bed active in the production of ethylene oxide per unit of tube volume available in a given tube reactor.

The selectivity (S) of the reacting to ethylene oxide (expressed in %) can be calculated according to the following equation (1):
Selectivity=100×(Molar production of ethylene oxide/hour)/(Molar consumption of ethylene/hour) (1)

The following examples illustrate the present invention.

### COMPARATIVE EXAMPLE 1

The manufacture of ethylene oxide was carried out continuously in a tube reactor (1) as shown in FIG. 1, comprising an inlet chamber (2), a central chamber (3) and an outlet chamber (4). The central chamber (3) comprised a bundle of reaction tubes (6) identical and parallel to one another and having a length L of 12 m. A silver-based catalyst was used, in the form of particles containing 14.7% by weight of silver deposited on an alumina support. The reaction tubes (6) were filled entirely with the particles of the catalyst in a non-diluted form (more particularly not diluted with particles of an inert solid).

There was introduced continuously into the tube reactor a reactive gas mixture current containing by volume 28.2% of ethylene, 6.5% of molecular oxygen, 5% of carbon dioxide, 4.7% of nitrogen, 5.5% of argon, 0.3% of ethane, 4.8 vpm of ethyl chloride and the remainder of methane, under an absolute pressure of 2.06 MPa, the reactive gas mixture current being pre-heated to about 150° C. Replenishment with fresh components of the reactive gas mixture current, more particularly of fresh ethylene and molecular oxygen, was carried out continuously to enable the composition of said current to be kept constant in the course of production. The temperature (T) (expressed in degrees Celsius) of the reactive gas mixture current was measured at certain points along the reaction tubes (6), so as to plot the temperature (T) as a function of the distance (D) (expressed in metres) separating the points of the measurement of the temperature (T) from the inlet (7) of the reaction tubes. It was found that the relation thus obtained could be represented by a curve similar to that (1) of the graph in FIG. 4.

Ethylene oxide was therefore manufactured under these conditions according to a production (Pr) of ethylene oxide (measured in tonnes of ethylene oxide per day), and the value of the selectivity (S) of the reaction to ethylene oxide (calculated according to equation (1) mentioned above) was determined, together with the value of the temperature (T₁₁) of the reactive gas mixture current in the reaction tubes at a distance of 11 m from the inlet (7) of the reaction tubes. The results of said calculations and measurements are given in Table 1.

It was observed that the profile of the temperature of the reactive gas mixture current was increasing constantly the whole length of the reaction tubes, more particularly in the zone situated towards the outlet of the reaction tubes and in particular in the final zone extending up to the outlet of the reaction tubes. It was found in addition that the temperature of the reactive gas mixture current at the outlet of the reaction tubes approached the flammability zone of the gas current, which created the danger that any reaction runway or any hot spot formed in said zone and combined with the increasing profile of the temperature would cause the explosion risks to rise enormously.

### EXAMPLE 2

Exactly the same procedure was adopted as in Comparative Example 1, except that a part of the particles of the catalyst was diluted with particles of alumina, as an inert solid, identical in nature, in shape and in mean size to those of the catalyst support used in the preparation of the catalyst. Each reaction tube (6) comprised, according to FIG. 2_{B}, a first zone Z1 starting from the inlet (7) of the reaction tube, extending over a length of 10.5 m and containing the catalyst in the form of non-diluted particles (more particularly not diluted with particles of an inert solid), then a second zone Z2, adjacent to the first zone Z1, extending over a length (X) of 1.5 in up to the outlet (8) of the reaction tube and containing the catalyst in the form of particles diluted with the particles of the alumina in a proportion (P) equal to 25 parts by volume per 100 parts by volume of the mixture resulting from the dilution of the particles of the catalyst with those of the alumina.

Under said conditions, ethylene oxide was manufactured and the same measurements and calculations as those made in Comparative Example 1 were carried out. In addition, the difference (ΔS) between the value of the selectivity (S) of the reaction to ethylene oxide obtained in the present example and that obtained in Comparative Example 1 was calculated. In the same manner, the difference (ΔT₁₁) between the value of the temperature (T₁₁) obtained in the present example and that obtained in Comparative Example 1 was calculated. The results of the measurements and calculations are given in Table 1.

It was found that despite a slight drop in the production (Pr) of ethylene oxide, the selectivity (S) of the reaction to ethylene oxide had increased compared with that obtained in Comparative Example 1 (ΔS=+1.1) and that the temperature (T₁₁) of the reactive gas current had diminished substantially compared with that obtained in Comparative Example 1 (ΔT₁₁−−6° C.). It was observed that the temperature of the reactive gas mixture current had exhibited a clearly decreasing profile in the zone situated towards the outlet of the reaction tubes and more particularly in the final zone extending up to the outlet of the reaction-tubes, according to a curve similar to that (2) of the graph of FIG. 4. Under said conditions, the temperature of the reactive gas mixture current at the outlet of the reaction tubes deviated substantially from the flammability zone of the gas current. As a result, the risk of the formation of hot spots and reaction runaways dropped significantly, and the safety of the process in terms more particularly of the explosion risks was improved substantially.

TABLE 1
Production (Pr) of ethylene oxide, ΔS of the selectivity (S)
of the reaction to ethylene oxide and ΔT₁₁ of the temperature T₁₁
of the reactive gas current, as a function of the proportion (P)
by volume of alumina in the diluted catalyst and of the length
(λ) of the zone Z2 of the dilution of the catalyst (according
to Comparative Example 1 and Examples 2 to 6).

| | P | | | | |
|---|---|---|---|---|---|
| | (parts by | λ | Pr | ΔS | ΔT₁₁ |
| Example | volume) | (m) | (tonnes/day) | (%) | (° C.) |
| 1 (comp) | 0 | — | 275 | — | — |
| 2 | 25 | 1.5 | 265 | +1.1 | −6 |
| 3 | 5 | 1.5 | 272 | +0.3 | −1.5 |
| 4 | 35 | 1.5 | 263 | +1.5 | −8 |
| 5 | 5 | 2.5 | 268 | +0.8 | −4 |
| 6 | 35 | 0.5 | 272 | +0.3 | −1 |

It was noticed in addition that by reason of the clearly decreasing temperature profile observed in the zone situated towards the outlet of the reaction tubes, a margin in the handling of the reaction temperature was now obtained which was substantially expanded with respect to the flammability zone of the gas current. Said margin was sufficient to allow the operating conditions of the process to be varied, and in particular to carry out, for example, an increase in the concentration of molecular oxygen in the reactive gas mixture current, so as to compensate easily at least in part for the slight loss in production of ethylene oxide noted in the present example compared with Comparative Example 1. This could be brought about advantageously without jeopardising the substantial increase in safety achieved in particular with respect to the flammability risks.

### EXAMPLE 3

Exactly the same procedure was adopted as in Example 2, except that the particles of the catalyst were diluted with the particles of the alumina in a proportion P equal to 5 parts by volume per 100 parts by volume of the mixture resulting from the dilution of the particles of the catalyst with those of the alumina (instead of 25 parts by volume).

Under said conditions ethylene oxide was manufactured and the same measurements and calculations as those carried out in Example 2 were made. The results of the measurements and calculations are given in Table 1.

It was found that despite a very slight drop in the production (Pr) of ethylene oxide, the selectivity (S) of the reaction to ethylene oxide had increased slightly compared with that obtained in Comparative Example 1 (ΔS=+0.3) and that the temperature (T₁₁) of the reactive gas mixture current had diminished compared with that obtained in Comparative Example 1 (ΔT₁₁=1.5° C.). It was observed that the temperature of the reactive gas mixture current had exhibited a slightly decreasing profile in the final zone of the reaction tubes extending up to the outlet of the reaction tubes. Hence the temperature of the reactive gas current at the outlet of the reaction tubes deviated from the flammability zone of the gas current. The risk of the formation of hot spots and reaction runaways dropped, so that the safety of the process in terms of the explosion risks was improved. It was noticed, as in Example 2, that the margin in the handling of the reaction temperature was now obtained in the present example in a sufficiently expanded manner to allow the very slight production loss to be compensated easily at least in part, without jeopardising the increase in safety achieved in particular in terms of the explosion risks.

### EXAMPLE 4

Exactly the same procedure was adopted as in Example 2, except that the particles of the catalyst were diluted with the particles of the alumina in a proportion P equal to 35 parts by volume per 100 parts by volume of the mixture resulting from the dilution of the particles of the catalyst with those of the alumina (instead of 25 parts by volume).

Under said conditions, ethylene oxide was manufactured and the same measurements and calculations as those carried out in Example 2 were made. The results of the measurements and calculations are given in Table 1.

It was found that despite a drop in the production (Pr) of ethylene oxide, the selectivity (S) of the reaction to ethylene oxide had increased substantially compared with that recorded in Comparative Example 1 (ΔS=+1.5) and that the temperature (T₁₁) of the reactive gas mixture current had diminished considerably compared with that recorded in Comparative Example 1 (ΔT₁₁=−8° C.). In addition, it was observed that the temperature of the reactive gas mixture current exhibited a substantially decreasing profile in the zone situated towards the outlet of the reaction tubes and more particularly in the final zone extending up to the outlet of the reaction tubes. Under said conditions, the temperature of the reactive gas mixture current at the outlet of the reaction tubes deviated considerably from the flammability zone of the gas current. The risk of the formation of hot spots and reaction runaways consequently dropped considerably, so that the safety of the process in terms of the explosion risks was very much improved. It was noticed, as in Example 2, that a margin in the handling of the reaction temperature was now obtained in a sufficiently expanded manner to allow the production loss to be compensated easily at least in part, without jeopardising the very substantial increase in safety achieved in particular in terms of the explosion risks.

### EXAMPLE 5

Exactly the same procedure was adopted as in Example 2, except that the second zone Z2 extends over a length (X) of 2.5 m (instead of 1.5 m) up to the outlet (8) of the reaction tubes, and that the particles of the catalyst are diluted with the particles of the alumina in a proportion P equal to 5 parts by volume per 100 parts by volume of the mixture resulting from the dilution of the particles of the catalyst with those of the alumina (instead of 25 parts by volume).

Under said conditions, ethylene oxide was manufactured and the same measurements and calculations as those carried out in Example 2 were made. The results of the measurements and calculations are given in Table 1.

It was found that despite a slight drop in the production (Pr) of ethylene oxide, the selectivity (S) of the reaction to ethylene oxide had increased substantially compared with that recorded in Comparative Example 1 (ΔS=+0.8) and that the temperature (T₁₁) of the reactive gas mixture current had diminished considerably compared with that recorded in Comparative Example 1 (ΔT₁₁=−4° C.). In addition, it was observed that the temperature of the reactive gas mixture current exhibited a substantially decreasing profile in the zone situated towards the outlet of the reaction tubes and more particularly in the final zone extending up to the outlet of the reaction tubes. Under said conditions, the temperature of the reactive gas mixture current at the outlet of the reaction tubes deviated very substantially from the flammability zone of the gas current. As a result, the risk of the formation of hot spots and reaction runaways decreased in a remarkable manner, and the safety of the process in terms of the explosion risks was very much improved. It was noticed, as in Example 2, that a margin in the handling of the reaction temperature was now obtained in a sufficiently expanded manner to allow the slight production loss to be compensated easily at least in part, without jeopardising the very substantial increase in safety achieved in particular in terms of the explosion risks.

### EXAMPLE 6

Exactly the same procedure was adopted as in Example 2, except that the second zone Z2 extends over a length (X) of 0.5 m (instead of 1.5 m) up to the outlet (8) of the reaction tubes, and that the particles of the catalyst were diluted with the particles of the alumina in a proportion P equal to 35 parts by volume per 100 parts by volume of the mixture resulting from the dilution of the particles of the catalyst with those of the alumina (instead of 25 parts by volume).

Under said conditions, ethylene oxide was manufactured and the same measurements and calculations as those carried out in Example 2 were made. The results of the measurements and calculations are given in Table 1.

It was found that despite a very slight drop in the production (Pr) of ethylene oxide, the selectivity (S) of the reaction to ethylene oxide had increased slightly compared with that recorded in Comparative Example 1 (ΔS=+0.3) and that the temperature (T₁₁) of the reactive gas current had decreased compared with that recorded in Comparative Example 1 (ΔT₁₁=−1° C.). For the remainder, the comments and conclusions are similar to those given for Example 3.

1. Process for manufacturing ethylene oxide by catalytic oxidation reaction of ethylene with molecular oxygen, said process comprising contacting a reactive gas mixture current comprising ethylene and molecular oxygen with a silver-based catalyst in the form of particles arranged as a fixed bed in reaction tubes combined as a bundle in a tube reactor, and being characterised in that the reactive gas mixture current flowing through the reaction tubes is contacted with the catalyst particles diluted with particles of an inert solid in a proportion increasing in the flow direction of said current.

2. Process according to claim 1, characterised in that the proportion of particles of the inert solid in the mixture resulting from the dilution of the particles of the catalyst with those of said solid increases continuously in the flow direction of the reactive gas mixture current.

3. Process according to claim 1, characterised in that the proportion of particles of the inert solid in the mixture resulting from the dilution of the particles of the catalyst with those of said solid increases discontinuously in the flow direction of the reactive gas mixture current.

4. Process according to claim 1, characterised in that the dilution of the particles of the catalyst with those of the inert solid is performed over the whole of the particles of the catalyst that are contained in the tubes.

5. Process according to claim 1, characterised in that the dilution of the particles of the catalyst with those of the inert solid is performed over a portion of the particles of the catalyst, said portion being arranged in a zone of the reaction tubes that is situated towards the outlet of the reaction tubes and more particularly extending up to the outlet of the reaction tubes, in the flow direction of the reactive gas mixture current.

6. Process according to claim 1, characterised in that the reactive gas mixture current flowing through the reaction tubes is contacted first of all with the catalyst in the form of non-diluted particles that are arranged in a first zone Z1 of the reaction tubes which is situated towards the inlet of the reaction tubes, then with the catalyst in the form of particles diluted with the particles of the inert solid in a constant proportion or one increasing in the flow direction of said current, the particles thus diluted being arranged in a second zone Z2 of the reaction tubes, adjacent to the first zone Z1 and situated towards the outlet of the reaction tubes, preferably extending up to the outlet of the reaction tubes.

7. Process according to claim 6, characterised in that the proportion of particles of the inert solid increases continuously or discontinuously in the flow direction of the reactive gas mixture current, over the whole of zone Z2 of the reaction tubes.

8. Process according to claim 6, characterised in that the zone Z2 of the reaction tubes starts in the last half of the length of the reaction tubes that is situated towards the outlet of the reaction tubes, preferably in the last third or the last quarter or else the last fifth of the length of the reaction tubes, situated towards the outlet of the reaction tubes, and at the latest before the thirtieth or preferably the last twenty-fifth of the length of the reaction tubes, situated towards the outlet of the reaction tubes.

9. Process according to claim 1, characterised in that the proportion of particles of the inert solid in the mixture resulting from the dilution of the particles of the catalyst with those of said solid is such that the number of parts by volume of the particles of the inert solid is chosen in a range of from 1 to 99 parts, preferably from 1 to 75 parts, in particular from 2 to 50 parts, more particularly from 2 to 40 parts per 100 parts by volume of said mixture.

10. Process according to claim 1, characterised in that the inert solid is chosen from among metals, metal alloys and refractory products.

11. Process according to claim 1, characterised in that the inert solid is chosen from among refractory oxides, refractory clays, ceramic products and glass type materials.

12. Process according to claim 1, characterised in that the catalyst is a silver-based supported catalyst, preferably comprising metallic silver deposited on a refractory solid support.

13. Process according to claim 12, characterised in that the inert solid is in the form of particles having a nature, a shape and a mean size similar to or in particular identical to those of the catalyst support.

14. Process according to claim 12, characterized in that the inert solid is chosen from among refractory products identical to or different in nature from that of the solid support used in the preparation of the catalyst.

15. Process according to claim 1, characterised in that the particles of the catalyst have a mean size chosen from a range extending from 1 to 20 mm, preferably from 3 to 12 mm, and are in particular in the form of spherical, hemispherical, spheroidal, cylindrical particles, of rings, pellets or granules.

16. Process according to claim 1, characterised in that the particles of the inert solid have a mean size chosen from a range extending from 1 to 20 mm, preferably from 3 to 12 mm, and are in particular in the form of spherical, hemispherical, spheroidal, cylindrical particles, of rings, pellets or granules.

17. Process according to claim 1, characterised in that the inert solid is in the form of particles such that a fixed bed formed with said particles exhibits a pressure loss identical to or preferably lower than that of a fixed bed that is identical but formed with the particles of the catalyst.

18. Process according to claim 1, characterised in that the heat exchange fluid in which the bundle of tubes is immersed is chosen from among water at saturation temperature under pressure and organic heat exchange fluids, in particular mixtures of oils or hydrocarbons.

19. Process according to claim 18, characterised in that the organic heat exchange fluid is used under a relative pressure extending from 100 to 1500 kPa, preferably from 200 to 800 kPa, more particularly from 200 to 600 kPa.

20. Process according to claim 18, characterised in that the water at saturation temperature under pressure is used under a relative pressure extending 1500 to 1800 kPa.

21. Process according to claim 1, characterised in that the temperature of the reactive gas mixture current in the reaction tubes is chosen from a range of from 140 to 350° C., preferably from 180 to 300° C., in particular from 190 to 280° C.

22. Process according to claim 1, characterised in that the reactive gas mixture current prior to flowing in the reaction tubes is pre-heated to a temperature of from 100 to 200° C., preferably from 140 to 190° C.

23. Process according to claim 1, characterised in that the temperature of the gas current resulting from the reaction at the outlet of the reaction tubes remains at a maximum temperature attained by the reactive gas current in the reaction tubes or preferably decreases to a temperature equal to or less than 250° C., preferably equal to or less than 240° C., in particular equal to or less than 230° C. and more particularly equal to or less than 220° C., in particular a temperature chosen in a range of from 180 to 250° C., preferably in a range of from 190 to 240° C., in particular from 200 to 230° C. and more particularly from 200 to 220° C.
